(19) United States
(12) Patent Application Publication
(10) Pub. No.: US 2012/0252834 A1
(43) Pub. Date: Oct. 4, 2012

(54) NOVEL USE AND METHOD OF RAPAMYCIN TO TREAT TOXIC SHOCK

(75) Inventors: Teresa Krakauer, Fort Detrick, MD (US); Marilyn Buckley, Fort Detrick, MD (US)

(73) Assignee: The Government Of United States, As Represented By The Secretary Of The Army

(21) Appl. No.: 13/387,299

(22) PCT Filed: Aug. 4, 2010
(86) PCT No.: PCT/US2010/044342

§ 371 Date: Jun. 11, 2012

Related U.S. Application Data
(60) Provisional application No. 61/231,348, filed on Aug. 5, 2009.

(51) Int. Cl. A61K 31/436; C07D 491/16; A61P 31/00

(52) U.S. Cl. 514/291; 546/90

(57) ABSTRACT

Rapamycin is used for prevention and treatment of toxic shock. The toxic shock is induced, for example, by a toxin, such as Staphylococcal enterotoxin A (SEA), Staphylococcal enterotoxin B (SEB), toxic shock syndrome toxin 1 (TSST-I), streptococcal pyrogenic exotoxin A (SPEA), and streptococcal pyrogenic exotoxin C (SPEC).

CROSS-REFERENCE TO RELATED PATENT APPLICATIONS

This application claims priority from U.S. Provisional Application No. 61/231,348, filed on Aug. 5, 2009, which is incorporated herein by reference in its entirety.

### STATEMENT REGARDING FEDERALLY SPONSORED RESEARCH OR DEVELOPMENT

Work related to the present invention had U.S. government support under Grant No. 3.10035_{—}07_RD-B, awarded by Defense Threat Reduction Agency to Teresa Krakauer. The government has certain rights in this invention.

### BACKGROUND OF THE INVENTION

The present invention relates to the treatment of toxic shock syndrome. More specifically, the invention relates to the utilization of rapamycin for treating toxic shock induced by staphylococcal and streptococcal exotoxins.

Documents cited in this description are denoted numerically, in parenthetical, by reference to a bibliography below.

Staphylococcal exotoxins are among the most common etiological agents that cause toxic shock syndrome (34-35, 44, 52). The disease is characterized by fever, hypotension, desquamation of skin, and dysfunction of multiple organ systems (8, 49). These toxins bind directly to the major histocompatibility complex (MHC) class II molecules on antigen-presenting cells and subsequently stimulate T cells expressing specific Vβ elements on T-cell receptors (9, 15, 25, 35, 41, 50). Staphylococcal enterotoxin B (SEB) and the distantly related toxic shock syndrome toxin 1 (TSST-1), are also called superantigens because they induce massive proliferation of T cells (35). In vitro and in vivo studies show that these superantigens induce high levels of various proinflammatory cytokines and these potent mediators cause lethal shock in animal models (1, 6, 23, 29, 39, 43, 46, 53, 58, 62).

SEB also causes food poisoning (4, 22, 59) and is a potential bioterrorism threat agent, as humans are extremely sensitive to this superantigen, especially by inhalation (34). There is currently no effective therapeutic treatment for SEB-induced shock except for the use of intravenous immunoglobulins (IVIG) (11). However IVIG is protective only when administered concurrently with SEB or soon after SEB exposure (32). Various in vitro experiments identified inhibitors to counteract the biological effects of SEB, only some of which were successful in ameliorating SEB-induced shock in experimental models (1, 26, 27, 29, 31, 58).

The gram-positive bacteria Staphylococcus aureus and Streptococcus pyogenes also produce a number of other superantigens which share a common three-dimensional structure and similar biological activities (16, 30, 45). Staphylococcal enterotoxin (SEA), TSST-1, streptococcal pyrogenic exotoxin A (SPEA) and streptococcal pyrogenic exotoxin C (SPEC) also bind to MHC class II molecules and specific Vβ elements on T-cell receptors on host cells (16, 36, 44). Many of the biological effects of these bacterial pyrogenic superantigens are similar in vitro and in vivo. The common structure and mechanism of action of bacterial superantigens often produce the same pathology and diseases.

In some prior studies, therapeutic agents had to be administered before SEB exposure to achieve protective effects (1, 33).

Rapamycin is a relatively new FDA-approved drug used to prevent graft rejection in renal transplantation, as it shows less nephrotoxicity than calcineurin inhibitors (7, 14, 47, 48, 56). Recent studies reveal other uses in animal models of cancer (24), diabetic nephropathy (42), bleomycin-induced pulmonary fibrosis (37), liver fibrosis (5) and tuberous sclerosis (38). Rapamycin binds intracellularly to FK506-binding proteins, specifically FKBP12, the rapamycin-FKBP12 complex then binds to a distinct molecular target called mammalian target of rapamycin (mTOR) (40, 56). Other studies identified the mTOR as the conserved serine-threonine kinase for sensing cellular stress and rapamycin promotes anabolic cellular processes in response to stress signals (21, 55, 57, 61). The mTOR pathway regulates myogenesis (13), cell cycle arrest (17, 21), adipocyte differentiation (3), and insulin signaling (55, 57). The immunological effects of rapamycin include regulation of T-cell activation (56), differentiation, expansion, and preservation of regulatory T cells (2, 10, 20, 54), downregulation of dendritic cells (12, 60), and GM-CSF-induced neutrophil migration (18).

There is a need to develop a treatment for toxic shock syndrome. In view of the potent immunosuppressive effects of rapamycin, the therapeutic impact of rapamycin on toxic shock is investigated.

### SUMMARY OF THE INVENTION

It is an object of the present invention to provide a new approach to treating toxic shock syndrome, particularly toxic shock induced by Staphylococcal exotoxins.

Accordingly, the present invention provides, in one aspect, a method for preventing or treating toxic shock by administering a therapeutically effective amount of an agent to a subject exposed to a toxin. In one embodiment, the agent is rapamycin.

In another embodiment, the toxic shock is induced by Staphylococcal enterotoxin A (SEA), Staphylococcal enterotoxin B (SEB), toxic shock syndrome toxin 1 (TSST-1), streptococcal pyrogenic exotoxin A (SPEA), or streptococcal pyrogenic exotoxin C (SPEC).

In yet another embodiment, rapamycin is administered to a subject in less than 24 hours, less than 23 hours, less than 22 hours, less than 21 hours, less than 20 hours, less than 19 hours, less than 18 hours, less than 17 hour, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour following exposure to a toxin.

In another embodiment, rapamycin is administered to a subject prior to exposure to a toxin. For example, rapamycin administered at 30 minutes to 1 hour prior to exposure to a toxin is expected to achieve the therapeutic effects.

In some embodiments, more than one dose of rapamycin is administered to a subject during a period of up to 96 hours following exposure to a toxin at an interval of every 24 hours. In one embodiment, the interval is every 12 hours, every 8 hours or every 4 hours. In another embodiment, the interval is every 3 hours or every 6 hours.

In a preferred embodiment, a first dose of rapamycin of 0.7 mg/kg is administered by IN, followed by daily dose of 1.6 mg/kg by IP until 96 hours. Preferably, the first dose of rapamycin is administered in less than 24 hours following exposure to a toxin. More preferably, the first dose of rapamycin is administered in less than 23 hours, less than 22 hours, less than 21 hours, less than 20 hours, less than 19 hours, less than 18 hours, less than 17 hour, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour.

In some embodiments, rapamycin is administered to a subject via gastrointestinal administration, (such as oral, gavage and rectal administrations) or via parenteral administration (such as intravenous, intramuscular, intranasal, intraperitoneal, and subcutaneous administrations).

Preferably, one or more doses of rapamycin are administered intranasally and additional one or more doses of rapamycin are administered intraperitoneally. More preferably, all doses of rapamycin are administered intranasally.

In a related aspect, the invention relates to use of rapamycin or a pharmaceutical composition comprising rapamycin for the treatment of toxic shock, particularly toxic shock induced by Staphylococcal enterotoxin A (SEA), Staphylococcal enterotoxin B (SEB), toxic shock syndrome toxin 1 (TSST-1), streptococcal pyrogenic exotoxin A (SPEA), or streptococcal pyrogenic exotoxin C (SPEC).

### BRIEF DESCRIPTION OF THE DRAWINGS

FIG. 1 demonstrates inhibition of TNFα, IL-1β, IL-6 (FIG. 1A), IL-2, IFNγ (FIG. 1B), and MCP-1, MIP-1α, MIP-1β (FIG. 1C) production by human PBMC stimulated with SEB alone or in the presence of various concentrations of rapamycin. Values represent the means±SD of duplicate samples from three experiments.

FIG. 2 demonstrates inhibition of T-cell proliferation in PBMC stimulated with SEB alone or in the presence of various concentrations of rapamycin. Values are the means±SD of triplicate cultures and represent three experiments.

FIG. 3 demonstrates inhibition of T-cell proliferation in PBMC stimulated with SEA, TSST-1, SPEA, SPEC alone or in the presence of 1 μg/mL of rapamycin. Values are the means±SD of quaduplicate cultures and represent two experiments.

FIG. 4 demonstrates rapamycin attenuation of the hypothermic response of C3H/HeJ mice treated with SEB. Body temperatures of mice exposed to BSA, SEB, SEB+rapamycin (0.7 mg/kg intranasal) at different time points after SEB exposure are shown. Rapamycin (1.6 mg/kg) was administered i.p. to all mice at 24, 48, 72, and 96 h. Mice received delayed treatment of rapamycin until 24 h also received i.p. doses of rapamycin (1.6 mg/kg) at 30, 48, 72, 96 h following i.n. rapamycin (0.7 mg/kg) at 24 h. Points represent the mean temperature±standard deviation (SD) for each group (n=10).

FIG. 5 demonstrates rapamycin prevention of weight loss in murine SEB-mediated shock model. Percentage mean weight data of mice exposed to SEB, SEB+rapamycin (0.7 mg/kg intranasal) at different time points after SEB are shown. Rapamycin (1.6 mg/kg) was administered i.p. to all mice at 24, 48, 72, and 96 h. Mice receiving delayed treatment of rapamycin until 24 h also received i.p. doses of rapamycin (1.6 mg/kg) at 30, 48, 72, 96 h following i.n. rapamycin (0.7 mg/kg) at 24 h. Points represent the % mean weight change for each group (n=10).

FIG. 6 demonstrates survival analysis of mice treated with (i) SEB, (ii) SEB+dexamethasone starting at 5 h (D5) post SEB exposure, (iii) SEB+rapamycin starting at 2 h (R2) post SEB, (iv) SEB+rapamycin starting at 3 h (R3) post SEB, (v) SEB+rapamycin starting at 4 h (R4) post SEB, (vii) SEB+rapamycin starting at 5 h (R5) post SEB. Time to death is in h after SEB exposure. For comparison, dexamethasone treatment (1.2 mg/kg) administered i.n. at 5 h after SEB, followed by dexamethasone (5 mg/kg) i.p. at 24, 48, 72 and 96 h after SEB was shown (D5).

FIG. 7 shows peak serum levels of MCP-1, IL-6 and IL-2 in mice (n=5) treated with SEB alone or SEB+rapamycin (0.7 mg/kg intranasal at 2 h). Values represent the mean±SD of duplicate samples. The “*” indicates P<0.05 when compared with mice treated with SEB.

FIG. 8 demonstrates rapamycin attenuation of the hypothermic response of C3H/HeJ mice treated with SEB using a shorter time course and a lower dose schedule. Body temperatures of mice exposed to SEB, SEB+rapamycin (0.4 mg/kg intranasal) at 5 h after SEB exposure are shown. Rapamycin group also received rapamycin (0.8 mg/kg) i.p. at 24, and 48 h (ds2). Points represent the mean temperature±standard deviation (SD) for each group (n=10).

FIG. 9 demonstrates rapamycin prevention of weight loss in murine SEB-mediated shock model using a shorter time course and a lower dose schedule. Percentage mean weight data of mice exposed to SEB, SEB+rapamycin (0.4 mg/kg intranasal) at 5 h after SEB exposure are shown. Rapamycin group also received rapamycin (0.8 mg/kg) i.p. at 24, and 48 h (ds2). Points represent the % mean weight change for each group (n=10).

FIG. 10 demonstrates rapamycin attenuation of the hypothermic response of C3H/HeJ mice treated with SEB using a lower dose and treatment schedule. Body temperatures of mice exposed to SEB, SEB+rapamycin (0.08 mg/kg intranasal) at 24 h after SEB exposure are shown. Rapamycin group also received rapamycin (0.3 mg/kg) i.p. at 30, 48, 72, and 96 h (ds3). Points represent the mean temperature±standard deviation (SD) for each group (n=10).

FIG. 11 demonstrates rapamycin prevention of weight loss in murine SEB-mediated shock model using a lower dose and treatment schedule. Percentage mean weight data of mice exposed to SEB, SEB+rapamycin (0.08 mg/kg intranasal) at 24 h after SEB exposure are shown. Rapamycin group also received rapamycin (0.3 mg/kg) i.p. at 30, 48, 72, and 96 h (ds3). Points represent the % mean weight change for each group (n=10).

FIG. 12 demonstrates rapamycin attenuation of the hypothermic response of C3H/HeJ mice treated with SEB using intranasal doses of rapamycin. Body temperatures of mice exposed to SEB, SEB+rapamycin (0.16 mg/kg intranasal) using 3 different treatment schedules after SEB exposure are shown. Rapamycin (0.16 mg/kg) was administered intranasally to mice at (i) 5, 24, 48, 72, and 96 h; (ii) 5, 24, 48, and 72 h; (iii) 17, 23, 41, 65, and 89 h. Points represent the mean temperature±standard deviation (SD) for each group (n=10).

FIG. 13 demonstrates rapamycin prevention of weight loss in murine SEB-mediated shock model using intranasal doses of rapamycin. Percentage mean weight data of mice exposed to SEB, SEB+rapamycin (0.16 mg/kg intranasal) using 2 different treatment schedules after SEB exposure are shown. Rapamycin (0.16 mg/kg) was administered intranasally to mice at (ii) 5, 24, 48, and 72 h; (iii) 17, 23, 41, 65, and 89 h. Points represent the % mean weight change for each group (n=10).

FIG. 14 demonstrates survival of mice treated with (i) SEB, (ii) SEB+rapamycin (0.16 mg/kg intranasal) at (i) 5, 24, 48, 72, and 96 h (Ri); (ii) 5, 24, 48, and 72 h (Rii); (iii) 17, 23, 41, 65, and 89 h (Riii). Time to death is in h after SEB exposure.

FIG. 15 demonstrates rapamycin prevents apoptosis of cells from C3H/HeJ mice treated with SEB. Fluorescent counts of cells from peripheral blood or spleen of mouse treated with DMSO (negative drug control), SEB and SEB+rapamycin (0.16 mg/kg intranasal) at 17 h after SEB exposure are shown. Cells were isolated from mice at 18 h after SEB exposure and stained with JC-1 dye. Flow cytometer was used to measure fluorescence and fluorescence data at 530 nm representing cellular apoptosis from peripheral blood or spleen taken from rapamycin treated and untreated mice are shown.

### DETAILED DESCRIPTION OF THE PREFERRED EMBODIMENTS

The present invention is rooted in the inventors' discovery of rapamycin's therapeutic effects in preventing and treating toxic shocks, especially toxic shocks induced by Staphylococcal exotoxins, such as Staphylococcal enterotoxin B-induced toxic shocks. The inventors demonstrate, for the first time, that rapamycin effectively inhibited SEB-mediated production of TNFα, IL-1β, IL-6, IL-2, IFNγ, MCP-1, MIP-1α, and MIP-1β by human PBMC in vitro. Besides decreasing the levels of proinflammatory cytokines in vitro, SEB-induced proliferation of T cells was also completely blocked by rapamycin. As excessive release of cytokines mediates the pathogenic effects of SEB in vivo, the use of rapamycin, an immunosuppressent was tested in vivo. For the first time, the present inventors demonstrate that rapamycin was 100% effective in protecting mice from SEB-mediated shock.

Previous studies of drug treatment for SEB-mediated shock models indicate a very narrow therapeutic window of treatment with different types of inhibitors to reduce the biological effects of SEB (1, 29, 30, 33, 58). For example, using the potent steroid dexamethasone, survival of mice in this model of SEB-induced toxic shock was 100%, 70%, 30%, 10% when mice were treated with dexamethasone at 2, 3, 4, 5 h, respectively, after SEB treatment (Table 1). As demonstrated in this application, rapamycin, provides even a wider therapeutic window, such that when given at up to 24 hours following SEB exposure, still afforded 100% protection against mortality, temperature, and weight fluctuations. Serum levels of MCP-1 and IL-6 were also markedly reduced when mice were treated with rapamycin compared to control SEB-exposed mice. These results reveal the potency of rapamycin even when given post SEB exposure. The serum cytokines, temperature, and weight data revealed the protective effects of rapamycin after SEB exposure. The high levels of IL-2 in vivo with rapamycin treatment appear to be transient and did not affect survival.

The rapamycin doses used in vivo in the present application are in the same range as those used in the field to reduce murine adjuvant arthritis and carcinogen-induced lung tumors (7, 19). Peak blood concentrations of rapamycin also agree with those used in murine models (19). In healthy subjects, blood concentrations of rapamycin reached to 78.2±18 ng/ml 1 h after 15 mg of rapamycin (47). The rapamycin dose used clinically to prevent graft rejection in renal transplantation is much lower (5 mg/day) with blood levels of 37.4±21 ng/ml at 1.8 h. However rapamycin is used for extended periods of years in these patients where long term accumulative toxic effects have to be taken into consideration. The doses in the present application are similar to those used in animal models of disease and the short course of treatment in this study suggests that this drug can be transition to human trials to treat SEB-induced shock. As such, a therapeutically effective amount of rapamycin via different administration routes for treating toxic shocks is within the purview of one of ordinary skill in the art. For example, an intranasal dose ranging from 0.08 mg/kg-0.7 mg/kg is effective; and an intraperitoneal dose ranging from 0.3 to 1.6 mg/kg is effective.

Because rapamycin has been approved by FDA and safely administered to critically ill transplant patients without significant toxicity even after 2 years of use, the application of rapamycin against toxic shock in humans is promising.

The following examples are given to illustrate the present invention. It should be understood, however, that the spirit and scope of the invention is not to be limited to the specific conditions or details described in these examples. All references identified herein are hereby expressly incorporated by reference.

### EXAMPLES

### Example 1

Materials and Methods

Reagents. Purified SEB was obtained from Toxin Technology (Sarasota, Fla.). The endotoxin content of these preparations was <1 ng of endotoxin/mg protein, as determined by the Limulus amoebocyte lysate assay (BioWhittaker, Walkersville, Md.). Human cytokine ELISA kits and assay reagents were purchased from R&D Systems (Minneapolis, Minn.). Mouse cytokine enzyme-linked immunosorbent assay (ELISA) reagents were obtained from Pharmingen (San Diego, Calif.). Rapamycin and all other reagents were from Sigma (St. Louis, Mo.). Rapamycin was prepared at 35 mg/ml in DMSO and diluted in saline prior to use.

Cell cultures. Human PBMC were isolated by Ficoll-Hypaque density gradient centrifugation of heparinized blood from normal human donors. The PBMC (10⁶ cells/ml) were cultured at 37° C. in 24-well plates containing RPMI 1640 medium and 10% heat-inactivated fetal bovine serum. Cells were stimulated with SEB (200 ng/ml) for 16 h and the supernatants were harvested and analyzed for TNFα, IL-1β, IL-6, IL-2, IFNγ, MCP-1, MIP-1α and MIP-1β by ELISA as described previously (26, 27). Rapamycin, when present, was added simultaneously with SEB.

Human T-cell proliferation assays. PBMC (10⁵ cells/well) were plated in triplicate with SEB (200 ng/ml), with or without varying concentrations of rapamycin, for 48 h at 37° C. in 96-well microtiter plates. Cells were pulsed with 1 μCi/well [³H]thymidine (New England Nuclear, Boston, Mass.) during the last 5 h of culture, as previously described (26). Cells were harvested onto glass fiber filters, and incorporated [³H]thymidine was measured by liquid scintillation.

Murine model of SEB-induced toxic shock. Male C3H/HeJ mice (National Cancer Institute, Frederick, Md.), weighing ˜20 g each (7-10 weeks old), were housed in conventional microisolator cages. Sterile temperature/identification transponders (IPTT-300, Biomedic Data Systems, Maywood, N.J.) were implanted subcutaneously into each animal 5-10 days before SEB, and temperatures were monitored twice daily. Initial weight of animals was recorded 3-7 days before SEB exposure and weight changes were recorded once daily after SEB challenge. SEB was administered intranasally (i.n.; 50 μl) with a micropipet and intraperitoneally (i.p.; 200 μl) with a tuberculin syringe (26G-3/8-inch needle). All intranasal doses were administered to mice previously anesthetized with an intramuscular (i.m.)-injected mixture of ketamine (2.4 mg/kg), acepromazine (0.024 mg/kg), and xylazine (0.27 mg/kg). There were 2 h of elapsed time between the first intranasal dose of 5 μg SEB/mouse, and the second i.p. dose of 2 μg SEB/mouse as this was the optimal time and dose previously determined to cause toxic shock without the use of synergistic agents (23). Mice exposed to both doses of SEB succumbed to death between 96-120 h and lethal endpoints were recorded up to 168 h after the first toxin dose. Controls consisted of C3H/HeJ mice given two doses of either bovine serum albumin (BSA, Sigma Chemical Corp.) or saline 2-h apart, similar to those of SEB-exposed mice (i.e., by i.n. and i.p. routes). For therapeutic investigations, mice (n=10) were given rapamycin i.n. at specific times after intranasal SEB exposure as described in each series of experiments. Intranasal rapamycin (0.7 mg/kg of body weight) in sterile saline (Sigma Chemical Corp. St. Louis, Mo.) was administered with a micropipet at the designated time points. This was followed by rapamycin (1.6 mg/kg) in sterile saline administered i.p. with a tuberculin syringe at 24, 48, 72, and 96 h after intranasal SEB. For comparison of effectiveness of therapeutics against SEB, a group of mice (n=10) received delayed treatment with i.n. rapamycin (0.7 mg/kg) at 24 h and subsequent i.p. doses at 30, 48, 72 and 96 h. Another group of mice (n=10) received delayed treatment of i.n. dexamethasone at 5 h followed by i.p. doses of dexamethasone at 24, 48, 72, 96 h after SEB. An alternative therapeutic protocol having shorter therapeutic period was intranasal rapamycin of 0.4 mg/kg at 5 h followed by rapamycin of 0.8 mg/kg via i.p. at 24, 48 h. A control lower dose protocol was intranasal rapamycin of 0.08 mg/kg at 24 h followed by rapamycin (0.3 mg/kg) via i.p. at 30, 48, 72, and 96 h. Other intranasal protocols were multiple doses of 0.16 mg/kg intranasal rapamycin at various time points and three treatment schedules as described in the legends of figures. Animals were monitored twice daily for illness and death for 96 h and as needed. Temperature and weight changes were measured daily up to 96-120 h. Temperature data were calculated as the mean temperature reading±standard deviation of each group (n=10 mice per group). The total number of mice dead versus alive was recorded at 168 h. Mice were followed for survival for 2-8 weeks.

Animal research was conducted in compliance with the Animal Welfare Act as well as other federal statutes and regulations. The facility where this research was conducted is fully accredited by the Association for Assessment and Accreditation of Laboratory Animal Care International. All efforts adhered to principles stated in the Guide for Care and Use of Laboratory Animals (National Research Council).

Previous results indicated the optimal timing for serum cytokine collection after mice were given a dual dose of SEB (23). Sera were collected from anesthetized mice by cardiac puncture at 5 and 24 h after SEB exposure. Rapamycin (0.7 mg/kg) was administered at 2 and 5 h after intranasal SEB. Serum cytokine/chemokine levels from individual mice (n=5 mice per time point), were analyzed for cytokines/chemokines using specific ELISA as described previously (23, 29, 45). Background levels of each cytokine/chemokine, all found to be negligible, were derived from a pre-bleed of the same mice performed 2-4 days before each experiment.

Determination of rapamycin in murine whole blood. Blood was collected from rapamycin-treated mice (n=5) by retro-orbital bleeding into EDTA tubes 1 h after intranasal administration of rapamycin. Blood was centrifuged at 12000×g for 3 min to remove cells. Supernatants was removed and stored at −70° C. until analysis. Analysis of rapamycin was performed as previously described (23) using an Agilent Technologies series 1100 Capillary HPLC instrument and ion-trap mass spectrometer.

Cytokine detection. The levels of human cytokines (TNFα, IL-1, IL-6, IL-2, IFNγ), and chemokines (MCP-1, MIP-1α, MIP-1β) in culture supernatants from PBMC, or murine mediators (TNFα, IL-1, IL-6, IL-2, IFNγ, MCP-1) in serum were measured via a sandwich ELISA by using cytokine-specific antibodies according to the manufacturer's instructions (23, 26, 27, 29, 46). Recombinant cytokines (20-1000 pg/ml) represented the standards for calibration and the detection limit of all assays was 20 pg/ml.

Detection of cellular apoptosis by measuring fluorescence of mitochondrial J-aggregates. The loss of mitochondrial permeability, indicative of cellular apoptosis was detected by a fluorescent cationic dye, JC-1 (51). Briefly, peripheral blood or spleens were removed at 18 h from mice treated with SEB, SEB plus 0.16 mg/kg of intranasal rapamycin at 17 h after SEB exposure. Negative controls consisted of mice treated with DMSO at 17 h after saline. Red blood cells from peripheral blood and spleens were lyzed with FACS lyzing buffer (Becton Dickinson, Mountainview, Calif.) prior to staining with JC-1 dye according to manufacturer's instructions (Immunochemistry Technology, Bloomington, Minn.). Flow cytometer (Becton Dickinson) was used to measure cell fluorescence at 530 nm, which represents apoptotic cells.

Statistical analysis. The cytokine and proliferation data were expressed as the mean±SD and analyzed for significant differences by the Student's t-test with Stata (Stata Corp., College Station, Tex.). Statistical comparisons of survival data were performed by Fisher's exact test with Stata software (Stata Corp.). Differences were considered significant if P was <0.05.

Results

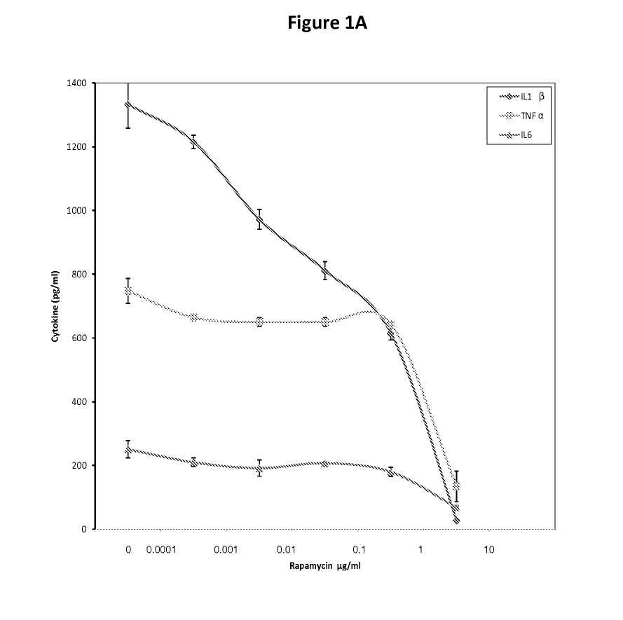

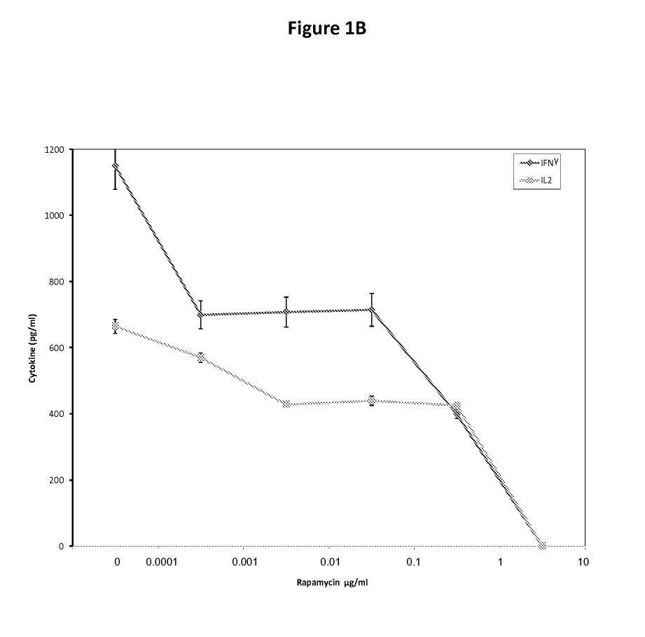

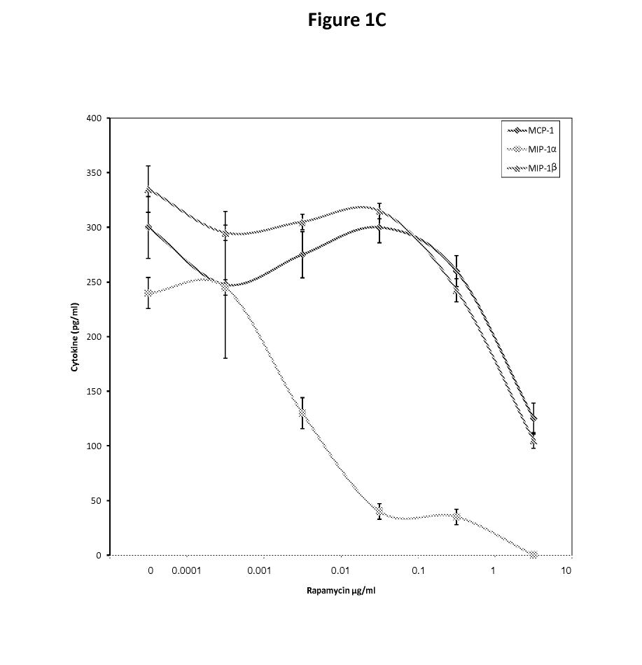

Rapamycin inhibits SEB-induced cytokines from human PBMC. Because proinflammatory cytokines mediate the lethal effects of SEB, the effect of rapamycin on the production of these mediators was examined on human PBMC incubated with SEB. FIG. 1 shows that rapamycin effectively blocked in a dose-dependent manner the production of TNFα, IL-1β, IL-6, IL-2, and IFNγ from PBMC incubated with SEB, achieving inhibition levels of 77%, 97%, 67%, 100%, respectively, at 6.9 μg/ml of rapamycin when compared to controls incubated with toxin alone (P<0.05). Rapamycin also dose dependently reduced the production of chemokines MCP-1, MIP-1α, and MIP-1β from SEB-stimulated PBMC. This indicates that rapamycin inhibited both cytokines and chemokines produced in vitro by both T cells and monocytes in response to SEB.

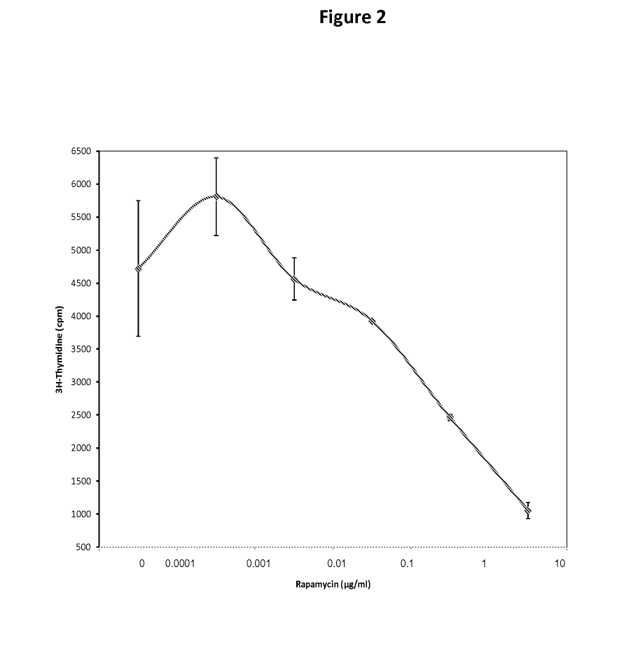

Rapamycin inhibits SEB-induced human T cell proliferation. In addition to increasing cytokine levels, SEB also stimulates T-cell proliferation. Therefore, the effect of rapamycin on SEB-induced proliferation of T cells was examined next. The results showed that rapamycin significantly decreased SEB-induced proliferation of T cells in a dose-response manner, with maximal inhibition (94%) achieved at the same concentration (6.9 μg/ml) that was most effective at blocking cytokine and chemokine release (FIG. 2). At 0.69 μg/ml rapamycin, T cell proliferation was reduced by 56%. The lack of proliferation or cytokine release from PBMC was not due to cytotoxic effect of rapamycin, as determined by Trypan Blue exclusion test (data not shown). Partial inhibition of proliferation was obtained when rapamycin was present for a short period of 3 h in cell culture. The effect of rapamycin was more pronounced on T-cell cytokines, IL-2 and IFNγ. At 6.9 ng/ml rapamycin, IL-2 and IFNγ was suppressed by 34% and 36% respectively, when compared to cells stimulated with SEB alone (P<0.05).

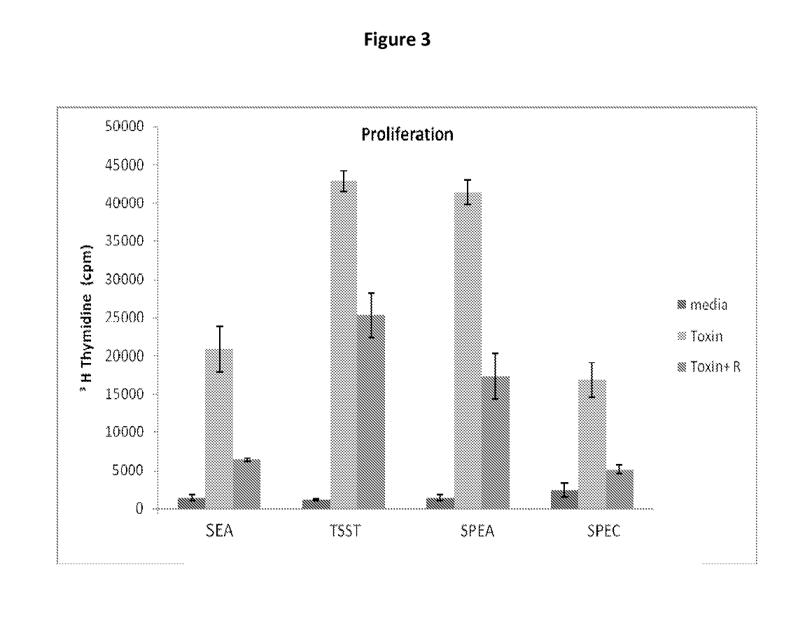

Rapamycin inhibits human T cell proliferation induced by other toxins. The effect of rapamycin in blocking the biological activities of other pyrogenic toxins, SEA, and TSST-1 from Staphyloccocus aureus and SPEA and SPEC from Streptococcus pyrogenes was examined in vitro. Rapamycin at 1 μg/mL reduced superantigen-induced T cell proliferation by 74%, 42%, 68% and 69% in SEA-, TSST-1-SPEA-, and SPEC-stimulated human peripheral blood mononuclear cells. Thus rapamycin has inhibitory activities towards a spectrum of bacterial pyrogenic toxins, as demonstrated in FIG. 3.

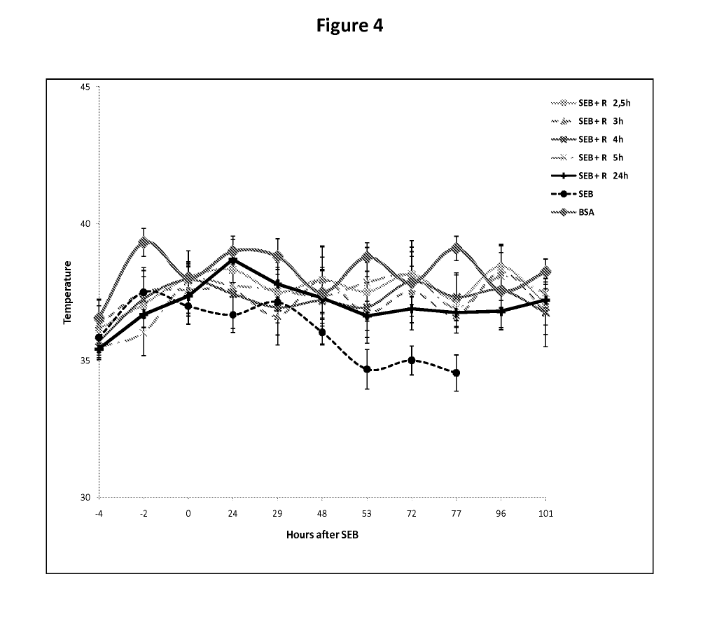

Rapamycin protects mice from SEB-mediated shock. Since rapamycin was effective in attenuating the biological effects of SEB in vitro, the efficacy of rapamycin in vivo was examined. Table 1 shows that rapamycin significantly increased the survival rate among mice given two doses of SEB. The time between the exposure of mice to SEB and rapamycin treatment was progressively increased. The mice were protected 100%, even when rapamycin was given 24 h after the SEB (Table 1). Clinical signs of intoxication such as ruffled fur and lethargy observed with SEB-treated mice starting at 72 h were completely absent from SEB+rapamycin group. Additional data were collected regarding temperature fluctuations in SEB-treated mice and SEB+rapamycin given at various times after SEB. Mice given SEB experienced hypothermia starting at 48 h (FIG. 4). This hypothermic response, indicating systemic shock that mimicked those found in other murine models, was completely absent in rapamycin-treated, SEB-exposed mice. Rapamycin protected mice from systemic shock even when it was administered 5 h after SEB, as seen from normal temperature of rapamycin-treated animals. When rapamycin treatment was delayed further, all mice (n=10) survived if rapamycin was administered by i.n. at 24 h, followed by i.p. doses at 30, 48, 72 and 96 h. These mice also maintained normal body temperature. However, delaying treatment with rapamycin to 32 h resulted in only 20% protection.

TABLE 1
Protective Effects of Rapamycin in vivo

| | % alive¹ | | | | | |
|---|---|---|---|---|---|---|
| | | Drug administered at | | | | |
| | No | 2 h and | | | | |
| Toxin | Drug | 5 h | 3 h | 4 h | 5 h | 24 h |
| SEB + rapamycin | 0 | 100 | 100 | 100 | 100 | 100 |
| SEB + | 0 | 100 | 70 | 30 | 10 | Not |
| dexamethasone | | | | | | determined |

¹The percentage of mice alive 168 h after intranasal SEB. Rapamycin (0.7 mg/kg) was given i.n. at the designated time points after SEB. All rapamycin-treatment groups (n = 10) also received rapamycin (1.6 mg/kg) i.p. 24, 48, 72 and 96 h after SEB. There were no lethal effects among mice given PBS or BSA. For comparison, dexamethasone treatment (1.2 mg/kg) administered i.n. at the designated time points after SEB, followed by dexamethasone (5 mg/kg) i.p. at 24, 48, 72 and 96 h after SEB.

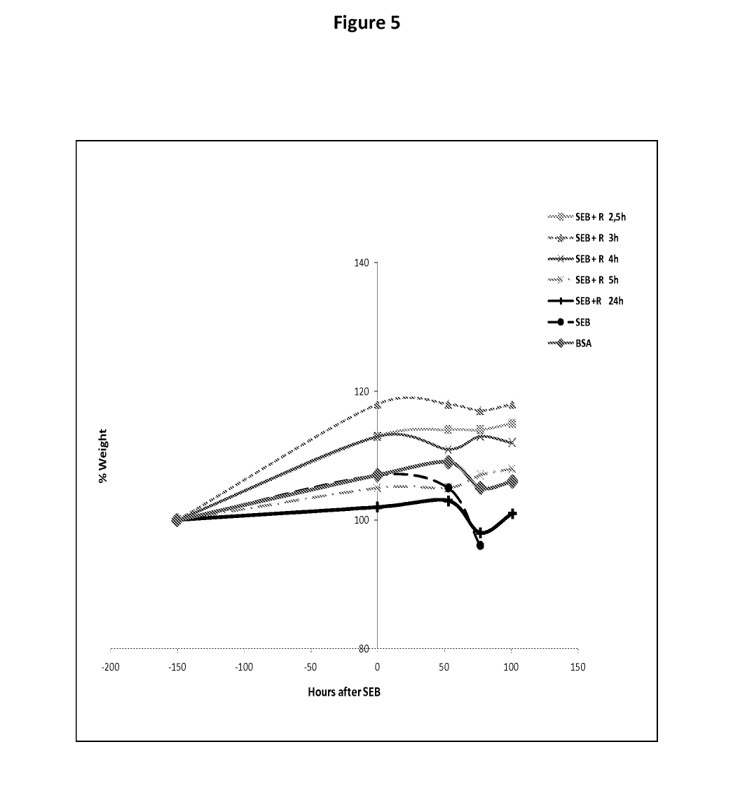

Weight loss is another prominent indicator of SEB-induced shock in other animal models of superantigen-induced disease (6, 39). The effect of rapamycin on the weight of animals after intranasal delivery of SEB was also examined. SEB-exposed mice experienced weight loss of 5% at 77 h whereas rapamycin-treated SEB-exposed mice gained weight of 8-17% over several days (FIG. 5). Rapamycin treatment ameliorated SEB-induced weight loss even when given 5 h after SEB. Protection against temperature and weight fluctuations essentially paralleled the lethality results.

Blood concentration of rapamycin in mice after oral dosing was determined by mass spectrophotometry to establish in vivo concentration for effective treatment as previously described (19). Blood level of rapamycin 1 h after an i.n. dose of 0.7 mg/kg was 492±204 ng/ml. At 97 h after multiple dosing (i.n. doses of 0.7 mg/kg at 2 and 5 h followed by i.p. doses of 1.6 mg/kg at 30, 48, 72 and 96 h), rapamycin reached to 622±282 ng/ml. The later concentration likely represented the highest concentration of rapamycin achieved in this dosing regiment as concentration of rapamycin reportedly peaked 1 h after dosing (47). In contrast, blood concentration of rapamycin at 97 h dropped to 38±15 ng/ml after using lower doses of rapamycin (0.03 mg/kg i.n. at 2 and 5 h and 0.08 mg/kg i.p. at 24, 48, 72 and 96 h) which were totally unprotective against SEB-mediated shock.

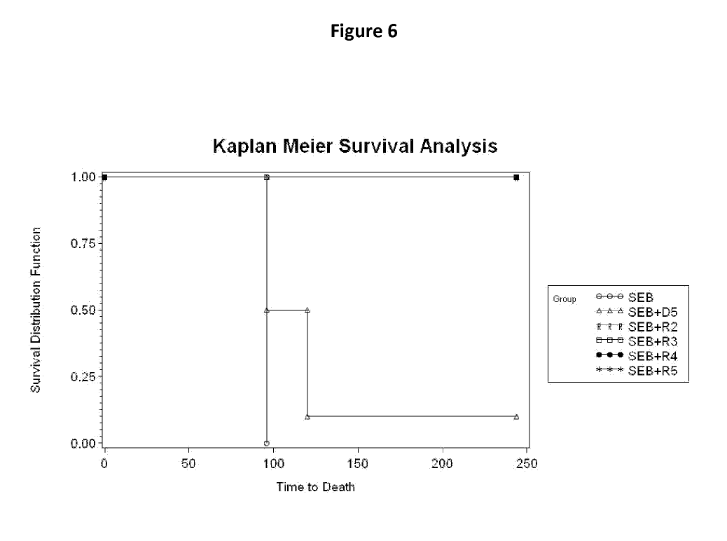

The efficacy of rapamycin against a potent immunosuppressant, the corticosteroid dexamethasone, was also compared. Dexamethasone was 100% effective in preventing lethality when mice (n=10) were treated with i.n. dexamethasone at 2 h and 5 h, followed by daily i.p. doses at 24, 48, 72, 96 h. However, delaying treatment with dexamethasone until 5 h after SEB exposure resulted in 90% lethality. Thus rapamycin is a more effective therapeutic post-SEB exposure than dexamethasone in vivo (Table 1 and FIG. 6). Blood level of rapamycin 1 h after i.n. dose of 0.7 mg/kg was 658±408 ng/ml. At 1 h after multiple dosing (2 i.n. doses plus 1 i.p. dose), rapamycin reached to 692±406 ng/ml. This later concentration likely represents the highest concentration of rapamycin achieved in the dosing regiment described as concentration of rapamycin peaks 1 h after dosing.

Rapamycin attenuates serum levels of IL-6 and MCP-1 in vivo. Based upon the strong inhibitory effects of rapamycin on SEB-mediated cytokine production and T-cell proliferation in vitro, the potential therapeutic role of rapamycin in vivo was further investigated in mice. Previous studies showed that elevated serum levels of MCP-1, IL-6 and IL-2 are a prominent feature of SEB-mediated toxic shock with intranasal delivery of SEB. Therefore, the in vivo effect of rapamycin on serum cytokine concentrations was examined in mice after SEB administration. Peak levels of MCP-1 and IL-6 were, respectively, reduced by 90% and 80% in SEB+rapamycin-treated mice versus SEB controls without rapamycin (FIG. 7). Serum concentrations of IL-2 remained unexplainably high in contrast to in vitro inhibitory effect of IL-2 by rapamycin in SEB-stimulated human PBMC. Serum concentrations of IL-2 remained high in contrast to in vitro inhibitory effect of IL-2 by rapamycin in SEB-stimulated human PBMC. Serum IL-2 concentrations at later time points were further examined. There was no IL-2 present at 24, 51, and 75 h in both SEB or SEB+rapamycin-treated mice (data not shown). Thus the elevated serum IL-2 at 5 h was transient with rapamycin treatment and did not affect lethality or the overall well-being of animals based on temperature and weight changes.

Since rapamycin was also effective when administered by i.n. at 24 h after SEB, lungs were [symbol] at 51 h post-SEB challenged for evaluation of the effect of rapamycin on lung cytokines Lung MCP-1, IL-2 and IL-6 were attenuated by 56%, 80%, and 65% respectively in mice treated with rapamycin when compared to SEB exposed group not treated with rapamycin.

### Example 2

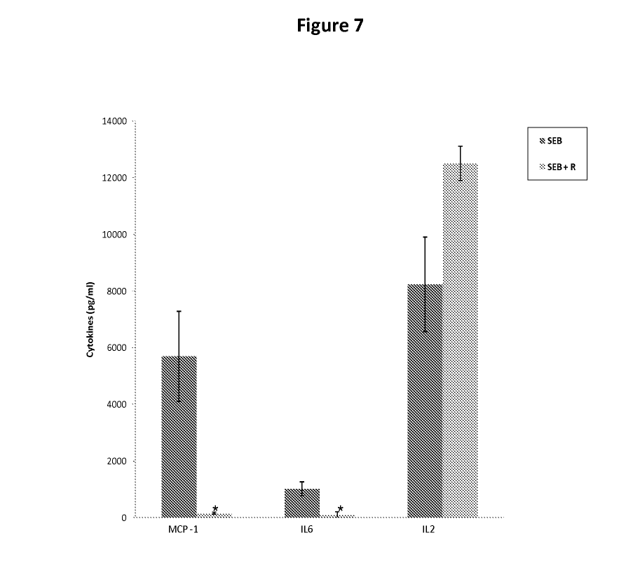

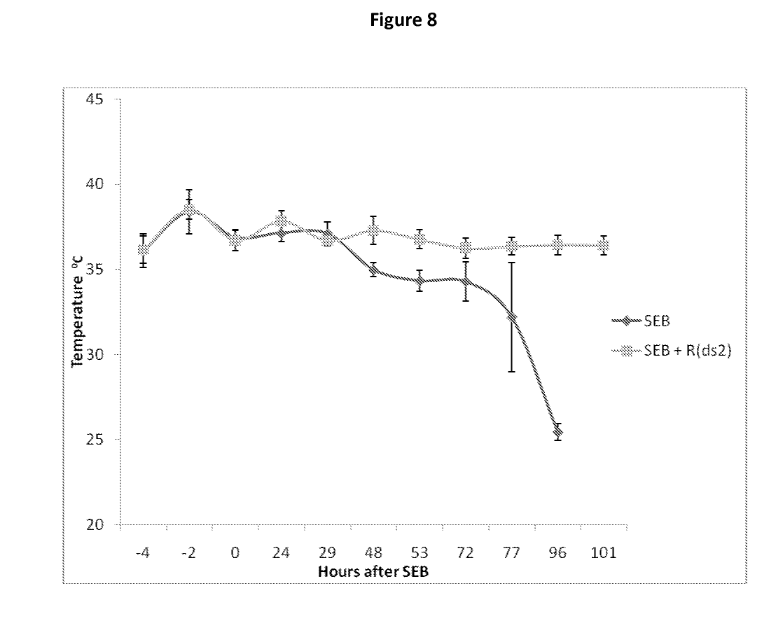

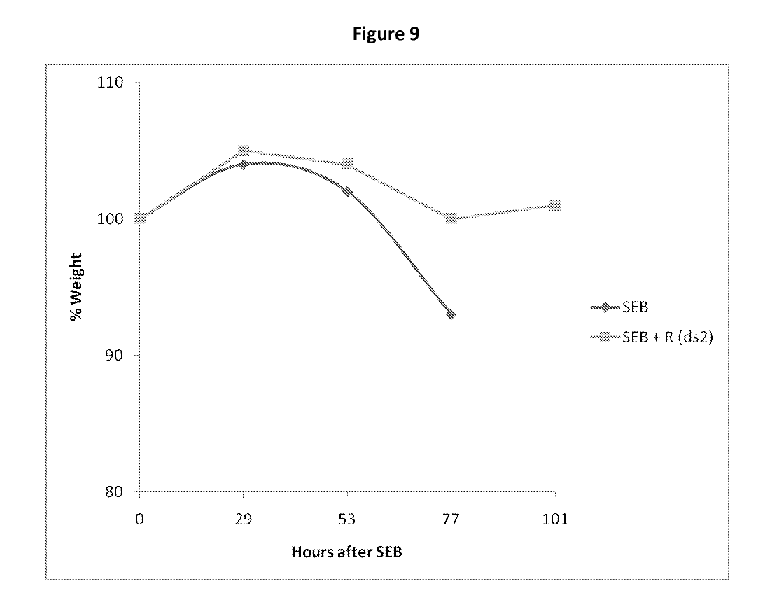

An initial dose of 0.4 mg/kg of rapamycin was administered intranasally to the mouse model of Example 1 at 5 hours following exposure to SEB, and additional doses at 0.8 mg/kg of rapamycin per dose were administered i.p. at 24 hours, and 48 hours following exposure to SEB. The results shown that mice were 100% protected and maintained normal body temperature and weight (FIGS. 8 and 9).

### Example 3

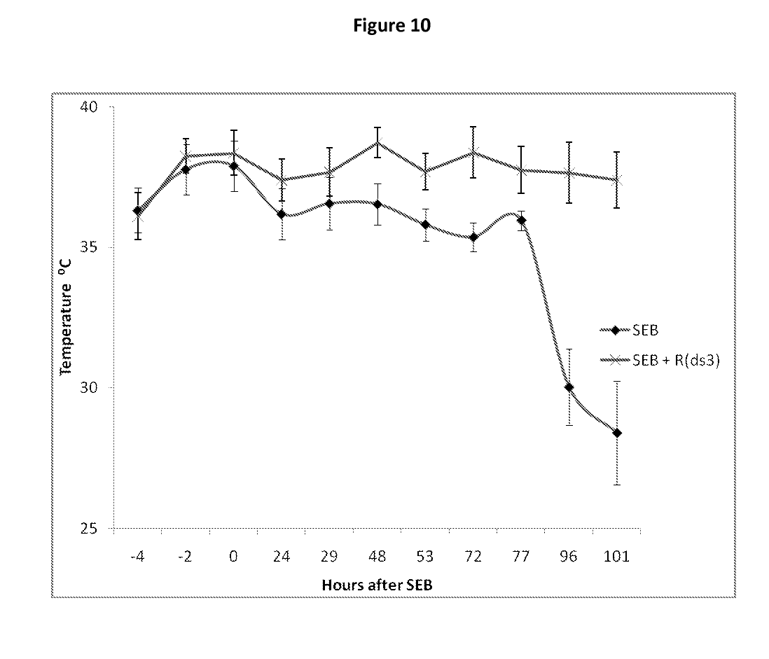

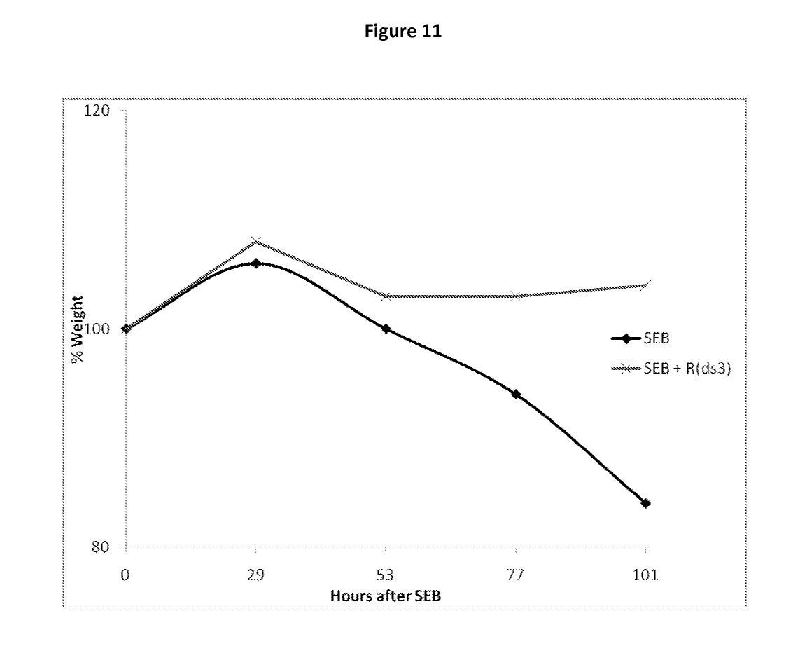

An initial dose of 0.08 mg/kg of rapamycin was administered intranasally to the mouse model of Example 1 at 24 hours following exposure to SEB, and additional doses at 0.3 mg/kg of rapamycin per dose were administered i.p. at 30 hours, 48 hours, 72 hours, and 96 hours following exposure to SEB. The results shown that mice were 100% protected and maintained normal body temperature and weight (FIGS. 10 and 11).

### Example 4

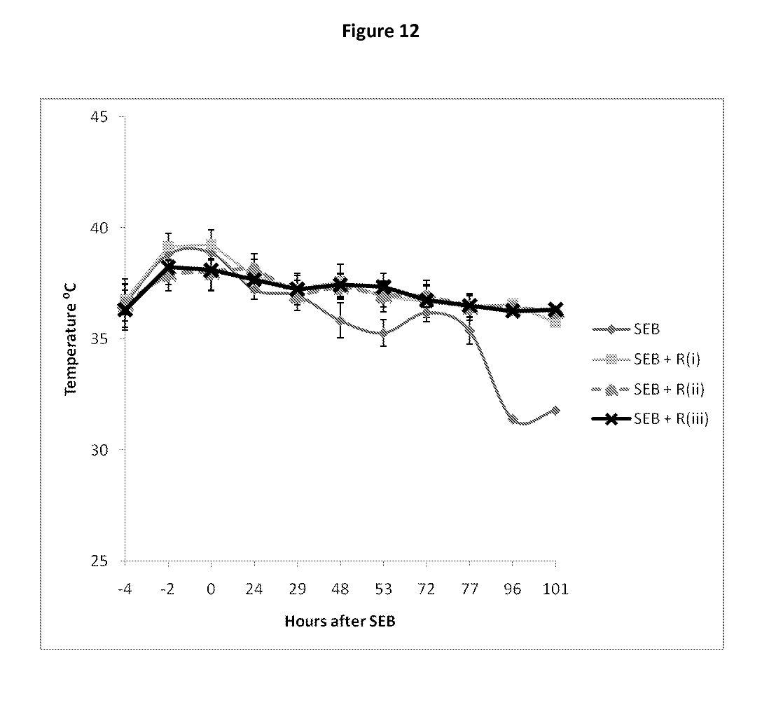

An initial dose of 0.16 mg/kg of rapamycin was administered intranasally to the mouse model of Example 1 at 5 hours following exposure to SEB, and additional doses at 0.16 mg/kg of rapamycin per dose were administered intranasally at 5 hours, 24 hours, 48 hours, 72 hours and 96 hours following exposure to SEB. The results shown that the mice were 100% protected and maintained normal body temperature (FIG. 12). Nasal application of rapamycin at lower doses is a distinct advantage for clinical application against toxic shock.

### Example 5

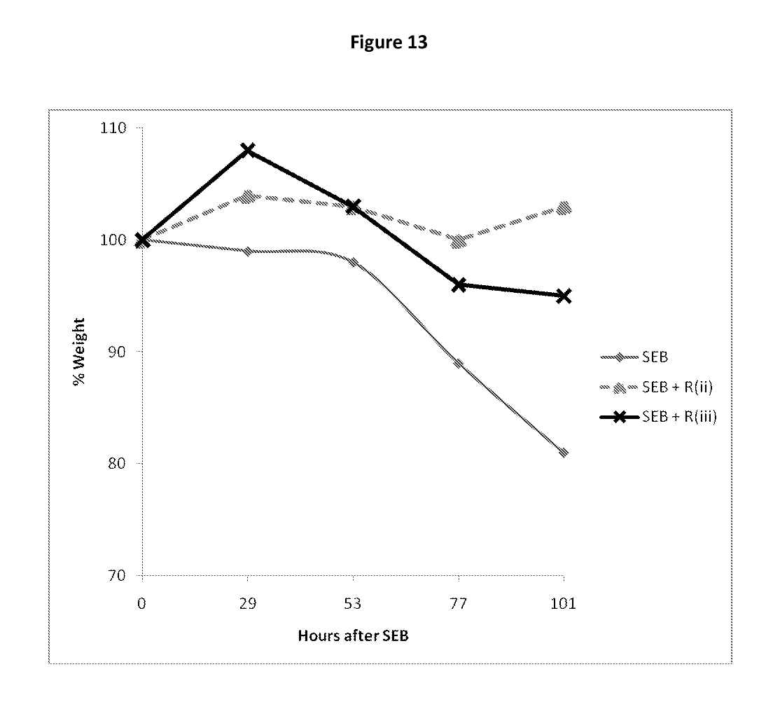

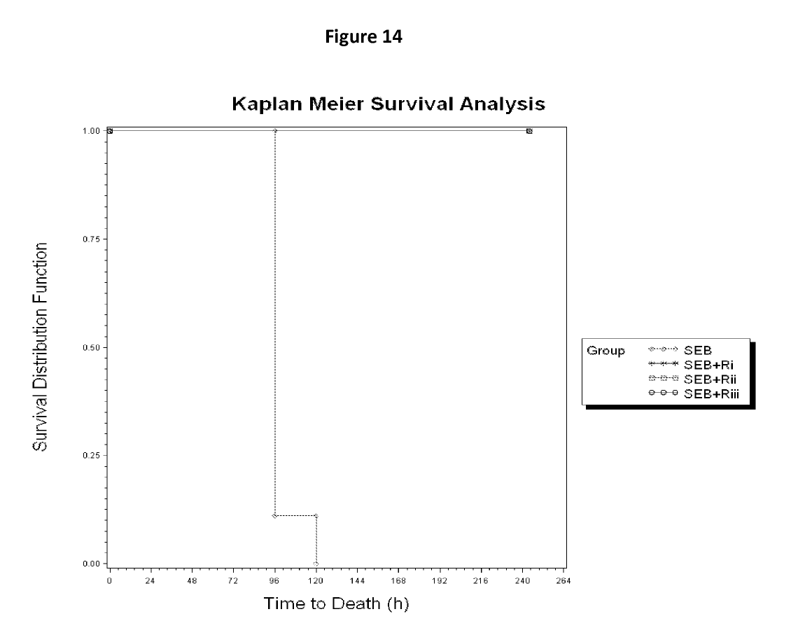

An initial dose of 0.16 mg/kg of rapamycin was administered intranasally to the mouse model of Example 1 at 5 hours following exposure to SEB, and additional doses at 0.16 mg/kg of rapamycin per dose were intranasally administered at 24 hours, 48 hours, and 72 hours following exposure to SEB. The results shown that the mice were 100% protected and maintained normal body temperature and weight (FIG. 12-14). Nasal application of rapamycin at lower doses and shorter course of treatment is a distinct advantage.

### Example 6

An initial dose of 0.16 mg/kg of rapamycin was administered intranasally to the mouse model of Example 1 at 17 hours following exposure to SEB, and additional doses at 0.16 mg/kg of rapamycin per dose were intranasally administered at 23 hours, 41 hours, 65 hours and 89 hours following exposure to SEB. The results shown that the mice were 100% protected and maintained normal body temperature and weight (FIG. 12-14). Nasal application of rapamycin at lower doses and wider window of treatment is a distinct advantage. Delayed initial administration to 17 hours provides better therapeutic window. When the initial dose of rapamycin was delayed to 24 hours, some level of protective effects was still achieved, as demonstrated in Table 2 below.

TABLE 2
Protective Effects of Intranasal Rapamycin in vivo

| | % alive¹ | | | | |
|---|---|---|---|---|---|
| | | Rapamycin administered at | | | |
| | No | 5, 24, 48, | 5, 24, 48, | 17, 23, 41, | 24, 30, 48, |
| Toxin | Drug | 72, 96 h | 72 h | 65, 89 h | 72, 96 h |
| SEB | 0 | 100% | 100% | 100% | 22% |

¹The percentage of mice alive 168 h after intranasal SEB. Rapamycin (0.16 mg/kg) was given i.n. at the designated time points after SEB. There were no lethal effects among mice (n = 10) given PBS or BSA since these mice were not exposed to SEB.

### Example 7

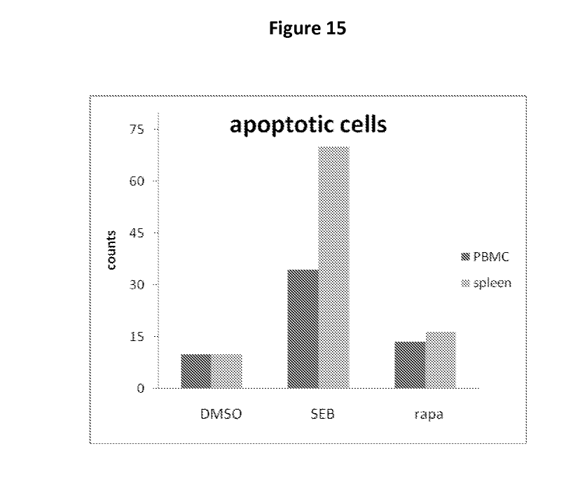

Other cellular events that might be contributed to rapamycin effects on SEB-induced shock were investigated in vivo. The fluorescent cationic dye, JC-1, was used to detect mitochondrial permeability transition events as an indicator of cell apoptosis. Both peripheral blood cells and splenocytes from mice treated with SEB for 18 h contained higher number of apoptotic cells. The number of apoptotic cells decreased with rapamycin treatment by 66% and 74% in mouse peripheral blood and spleen, respectively (FIG. 15). These results indicate that rapamycin promotes cell survival after SEB exposure.

### CITED PUBLICATIONS

1. Arad, G., R. Levy, D. Hillman, and R. Kaempfer. 2000. Superantigen antagonist protects against lethal shock and defines a new domain for T-cell activation. Nat. Med. 6:414-421.
2. Battaglia, M., A. Stabilini, and M. G. Roncarolo. 2005. Rapamycin selectively expands CD4⁺CD25⁺FoxP3⁺ regulatory T cells. Blood. 105: 4743-4748.
3. Bell, A., L. Grunder, A. Sorisky. 2000. Rapamycin inhibits human adipocyte differentiation in primary culture. Obes. Res. 8:249-254.
4. Bergdoll, M. S. 1988. Monkey feeding test for staphylococcal enterotoxin. Meth. Enzymol. 165:324-333.
5. Biecker, E., A. DeGottardi, M. Neef, M. Unternahrer, V. Schneider, M. Ledermann, H. Sagesser, S. Shaw, R. Reichen. 2005. Long-term treatment of bile duct-ligated rats with rapamycin (sirolimus) significantly attenuates liver fibrosis: analysis of the underlying mechanisms. J. Pharm. Exp. Ther. 313:952-961.
6. Blank, C., A. Luz, S. Bendigs, A. Erdmann, H. Wagner, and K. Heeg. 1997. Superantigen and endotoxin synergize in the induction of lethal shock. Eur. J. Immunol. 27:825-833.
7. Blazar, B., R. P. A. Taylor, S. N. Sehgal, and D. A. Vallera. 1994. Rapamycin, a potent inhibitor of T cell function, prevents graft rejection in murine recipients of allogeneic T-cell depended donor marrow. Blood. 83:600-609.
8. Chesney, P. J., J. P. Davis, W. K. Purday, P. J. Wand, and R. W. Chesney. 1981. Clinical manifestations of toxic shock syndrome. J. Am. Med. Assoc. 246:741-748.
9. Choi, Y., B. Kotzin, L. Hernon, J. Callahan, P. Marrack, and J. Kappler. 1989. Interaction of Staphyloccocus aureus toxin “superantigens” with human T cells. Proc. Natl. Acad. Sci. USA. 86:8941-8945.
10. Coenen, J. J., H. J. Koenen, E. van Rijssen, L. B. Hilbrands, and I. Joosten. 2006. Rapamycin, and not cyclosporin A, preserves the highly suppressive CD27⁺ subset of human CD4⁺CD25⁺ regulatory T cells. Blood. 107, 1018-1023.
11. Darenberg, J., B. Soderquist, B. H. Normark, and A. A. Norrby-Tegland. 2004. Differences in potency of intravenous polyspecific immunoglobin G against streptoccocal and staphyloccocal superantigen: implications for therapy of toxic shock syndrome. Clin. Infect. Dis. 38:836-842.
12. Das, S., A. Haddadi, S. Veniamin, and J. Samuel. 2008. Delivery of rapamycin-loaded nanoparticle down regulates ICAM-1 expression and maintains an immunosuppressive profile in human CD34⁺ progenitor-derived dendritic cells. J. Biomed. Mater. Res. A. 85:983-992.
13. Erbay E. and J. Chen. 2001. The mammalian target of rapamycin regulates C2C12 myogenesis via a kinase-independent mechanism. J. Biol. Chem. 276:36079-36082.
14. Flechner, S. M. 2009. Sirolimus in kidney transplantation indications and practical guidelines: de novo sirolimus-based therapy without calcineurin inhibitors. Transplantation. 87:S1-6.
15. Fleischer, B., H. Schrezenmeier, and P. Conradt. 1989. T lymphocyte activation by staphylococcal enterotoxins: role of class II molecules and T cell surface structures. Cell Immunol. 120:92-101.
16. Fraser, J. D., and T. Proft. 2007. The Streptococcal superantigens. In Superantigens: Molecular basis for their role in human diseases. Kotb, M.; Fraser, J. D., Ed; ASM Press, Washington, D.C. pp 3-20.
17. Frost, P., Y. Shi, B. Hoang, J. Gera, and A. Lichtenstein. 2009. Regulation of D-cyclin translation inhibition in myeloma cells treated with mammalian target of rapamycin inhibitors: rationale for combined treatment with extracellular signal-regulated kinase inhibitors and rapamycin. Mol. Cancer Ther. 8:83-93.
18. Gomez-Cambronero, J. 2003. Rapamycin inhibits GM-CSF-induced neutrophil migration. FEBS Lett. 550:94-100.
19. Granville, C. A., N. Warfel, J. Tsurutani, M. C. Hollander, et. al. 2007. Identification of a highly effective rapamycin schedule that markedly reduces the size, multiplicity, and phenotypic progression of tobacco carcinogen-induced murine lung tumors. Clin. Cancer Res. 13:2281-2289.
20. Haxhinasto, S., D. Mathis, and C. Benoist. 2008. The AKT-mTOR axis regulates de novo differentiation of CD4⁺Foxp3⁺ cells. J. Exp. Med. 205:565-574.
21. Heitman, J., N. R. Movva, and M. N Hall. 1991. Targets for cell cycle arrest by the immunosuppressant rapamycin in yeast. Science. 253:905-909.
22. Holmberg, S. D, and P. A. Blake. 1984. Staphylococcal food poisoning in the United States. New facts and old misconceptions. J. Am. Med. Assoc. 251:487-489.
23. Huzella, L. M., M. J. Buckley, D. A. Alves, B. G. Stiles, and T. Krakauer. 2009. Central roles for IL-2 and MCP-1 following intranasal exposure to SEB: A new mouse model. Res. Vet. Sci. 86:241-247.
24. Koehl, G. E., J. Andrassy, M. Guba, S. Richter, A. Kroemer, M. N. Scherer, M. Steinbauer, C. Graeb, H. J. Schlitt, K.-W. Jauch, E. K. Geissler. 2004. Rapamycin protects allografts from rejection while simultaneously attacking tumors in immunosuppressed mice. Transplantation. 77:1319-1326.
25. Kotzin, B. L., D. Y. M. Leung, J. Kappler, and P. A. Marrack. 1993. Superantigens and their potential role in human disease. Adv. Immunol. 54:99-166.
26. Krakauer, T. 1994 Inhibition of toxic shock syndrome toxin-induced cytokine production and T cell activation by interleukin 10, interleukin 4, and dexamethasone. J. Infect. Dis. 172:988-992.
27. Krakauer, T. 1999. The induction of CC chemokines in human peripheral blood mononuclear cells by staphylococcal exotoxins and its prevention by pentoxifylline. J. Leukco. Biol. 66:158-164.
28. Krakauer, T. 1999. Immune response to Staphylococcal superantigens. Immunol. Res. 20:163-173.
29. Krakauer, T. and M. Buckley. 2006. Dexamethasone attenuates Staphylococcal Enterotoxin B-induced hypothermic response and protects mice from superantigen-induced toxic shock. Antimicrob. Agents Chemother. 50:391-395.
30. Krakauer, T. 2007. Small nonpeptide inhibitors of Staphylococcal superantigen-induced cytokine production and toxic shock. In Superantigens: Molecular basis for their role in human diseases. Kotb, M.; Fraser, J. D., Ed; Asm Press, Washington, D.C. pp 229-224.
31. Krakauer, T., J. Stephens, M. Buckley, and M. Tate. 2007. Superantigen-induced cytokine release from whole-blood cell culture as a functional measure of drug efficacy after oral dosing in nonhuman primates. Res. In Vet. Sci. 83:182-187.
32. Leclaire, R. D., R. E. Hunt, and S. Bavari. 2002. Protection against bacterial superantigen staphylococcal enterotoxin B by passive vaccination. Infect. Immun. 70:2278-2281.
33. Levy, R., I. Nasie, D. Hillman, G. Arad, and R. Kaempfer. 2007. Broad-spectrum peptide antagonists of superantigen toxins. In Superantigens: Molecular basis for their role in human diseases, Kotb, M.; Fraser, J. D., Ed; ASM Press, Washington, D.C. pp 217-227.
34. Madsen, J. M. 2001. Toxins as weapons of mass destruction. A comparison and contrast with biological-warfare and chemical-warfare agents. Clin. Lab. Med. 21:593-605.
35. Marrack, P., J. Kappler. 1990. The staphylococcal enterotoxins and their relatives. Science. 248:705-709.
36. McCormick, J. K., J. M. Yarwood, and P. M. Schlievert. 2001. Toxic shock syndrome and bacterial superantigens: an update. Ann Rev. Microbiol. 55:77-104.
37. Mehrad, B., M. D. Burdick, and R. M. Strieter. 2009. Fibrocyte CXCR4 regulation as a therapeutic target in pulmonary fibrosis. Int. J. Biochem. Cell Bio. 41:1708-1718.
38. Meikle, L., K. Pollizzi, A. Egnor, I. Kramvis, H. Lane, M. Sahin, and D. J. Kwiatkowski. 2008. Response of a Neuronal Model of Tuberous Sclerosis to Mammalian Target of Rapamycin (mTOR) Inhibitors: Effects on mTORC1 and Akt Signaling Lead to Improved Survival and Function. J. Neurosci. 28:5422-5432.
39. Miethke, T., C. Wahl, K. Heeg, B. Echtenacher, P. H. Krammer, and H. Wagner. 1992. T cell-mediated lethal shock triggered in mice by the superantigen SEB: critical role of TNF. J. Exp. Med. 175:91-98.
40. Mita, M. M., A. Mita, and E. K. Rowinsky. 2003. The molecular target of rapamycin (mTOR) as a therapeutic target against cancer. Cancer Biol. Ther. 2:s169-77.
41. Mollick, J. A, M. Chintagumpala, R. G. Cook, and R. R. Rich. 1991. Staphylococcal exotoxin activation of T cells. Role of exotoxin-MHC class II binding affinity and class II isotype. J. Immunol. 146:463-468.
42. Mori, H., K. Inoki, K. Masutani, Y. Wakabayashi, K. Komai, R. Nakagawa, K. L. Guan, and A. Yoshimura. 2009. The mTOR pathway is highly activated in diabetic nephropathy and rapamycin has a strong therapeutic potential. Biochem. Biophy. Res. Comm. 384:471-475.
43. Nagaki, M., Y. Muto, H. Ohnishi, S. Yasuda, K. Sano, T. Naito, T. Maeda, T. Yamada, and H. Moriwaki. 1994. Hepatic injury and lethal shock in galactosamine-sensitized mice induced by the superantigen staphylococcal enterotoxin B. Gastroenterology. 106:450-458.
44. Proft, T., and J. D. Fraser. 2003. Bacterial superantigens. Clin. Exp. Immunol. 133:299-306.
45. Proft, T., S. Sriskandan, L. Yang, and J. D. Fraser. 2003. Superantigens and Streptococcal toxic shock syndrome. Emerg. Infect. Dis. 9:1211-1218.
46. Roy, C. J., K. L. Warfield, B. C. Welcher, R. F. Gonzales, T. Larsen, J. Hanson, C. S. David, T. Krakauer, and S. Bavari. 2005. Human leukocyte antigen-DQ8 transgenic mice: a model to examine the toxicity of aerosolized staphylococcal enterotoxin B. Infect. Immun. 73:2452-2460.
47. Saunders, R. N., M. S. Metcalfe, and M. L. Nicholson. 2001. Rapamycin in transplantation: a review of the evidence. Kidney Int. 59:3-16.
48. Sehgal, S. N. 2003. Sirolimus: its discovery, biological properties, and mechanism of action. Transplant Proc. 35:7S-14S.
49. Schlievert, P. M. 1993. Role of superantigens in human disease. J. Infect. Dis. 167:997-1002.
50. Scholl, P., A. Diez, W. Mourad, J. Parsonnet, R. S. Geha, and T. Chatila. 1989. Toxic shock syndrome toxin-1 binds to major histocompatibility complex class II molecules. Proc. Natl. Acad. Sci. USA. 86:4210-4214.
51. Smiley, S. T., M. Reers, C. Momottola-Hartshorn, M. Lin, A. Chen, T. W. Smith, G. D. Steele, and L. B. Chen. 1991. Intracellular heterogeneity in mitochondrial membrane potentials revealed by a J-aggregate forming lipophilic cation JC-1. Proc. Nat. Acad. Sci. USA. 88:3671-3675.
52. Stevens, D. L. 1996. The toxic shock syndromes. Infect. Dis. Clin. North Am. 10:727-746.
53. Stiles, B. G., S. Bavari, T. Krakauer, and R. G. Ulrich. 1993. Toxicity of staphylococcal enterotoxins potentiated by lipopolysaccharide: major histocompatibility complex class II molecule dependency and cytokine release. Infect. Immun. 61:5333-5338.
54. Strauss L., T. L. Whiteside, A. Knights, C. Bergmann, A. Knuth, A. Zippelius. 2007. Selective survival of naturally occurring human CD4+CD25+Foxp3+ regulatory T cells cultured with rapamycin. J. Immunol. 178:320-329.
55. Takano, A., I. Usui, T. Haruta, J. Kawahara, T. Uno, M. Iwata, and M. Kobayashi. 2001. Mammalian target of rapamycin pathway regulates insulin signaling via subcellular redistribution of insulin receptor substrate 1 and integrates nutritional signals and metabolic signals of insulin. Mol. Cell. Biol. 21:5050-5062.
56. Thomson, A. W., H. R. Turnquist, and G. Raimondi. 2009. Immunoregulatory functions of mTOR inhibition. Nature Rev. Immun. 9:324-37.
57. Tremblay, F., A. Gagnon, A. Veilleux, A. Sorisky, and A. Marette. 2004. Activation of the mammalian target of rapamycin pathway acutely inhibits insulin signaling to Akt and glucose transport in 3T3-L1 and human adipocytes. Endocrinology. 146:1328-37.
58. Visvanathan, K., A. Charles, J. Bannan, P. Pugach, K. Kashfi, and J. B. Zabriskie. 2001. Inhibition of bacterial superantigens by peptides and antibodies. Infect. Immun. 69:875-884.
59. Wieneke, A., D. Roberts, and R. J. Gilbert. 1993. Staphylococcal food poisoning in the United Kingdom, 1969-1990. Epidemiol. Infect. 110:519-531.
60. Woltman, A. M., S. W. van der Kooij, P. J. Coffer, R. Offringa, M. R. Daha, and C. van Kooten. 2003. Rapamycin specifically interferes with GM-CSF signaling in human dendritic cells, leading to apoptosis via increased p27^{KIP1} expression. Blood. 101:1439-1445.
61. Wullschleger, S., R. Loewith, and M. N. Hall. 2006. TOR signaling in growth and metabolism. Cell. 124:471-484.
62. Yeung, R. S., J. M. Penninger, T. Kundig, W. Khoo, P. S. Ohashi, G. Kroemer, and T. W. Mak. 1996. Human CD4 and human major histocompatibility complex class II (DQ6) transgenic mice: supersensitivity to superantigen-induced septic shock. Eur. J. Immunol. 26:1074-1082.

What is claimed is:

1. A method for preventing or treating toxic shock in a subject, comprising administering to said subject a therapeutically effective amount of rapamycin.

2. The method of claim 1, wherein the toxic shock is induced by exposure to a toxin.

3. The method of claim 2, wherein the toxin is a Staphylococcal exotoxin.

4. The method of claim 3, wherein the Staphylococcal exotoxin is Staphylococcal enterotoxin B.

5. The method of claim 2, wherein the toxin is selected from the group consisting of Staphylococcal enterotoxin A (SEA), Staphylococcal enterotoxin B (SEB), toxic shock syndrome toxin 1 (TSST-1), streptococcal pyrogenic exotoxin A (SPEA), and streptococcal pyrogenic exotoxin C (SPEC).

6. The method of claim 2, wherein rapamycin is administered in less than 24 hours, less than 23 hours, less than 22 hours, less than 21 hours, less than 20 hours, less than 19 hours, less than 18 hours, less than 17 hour, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour after exposure.

7. The method of claim 2, wherein more than one dose of rapamycin is administered to the subject during a period of up to 96 hours after exposure.

8. The method of claim 7, wherein rapamycin is administered at an interval of every 3 hours or every 6 hours.

9. The method of claim 7, wherein the first dose of rapamycin is administered intranasally.

10. The method of claim 7, wherein the additional doses of rapamycin after the first dose is administered intraperitoneally.

11. The method of claim 7, wherein all doses of rapamycin are administered intranasally.

12. The method of claim 7, wherein the first dose of rapamycin is administered in less than 24 hours after exposure.

13. The method of claim 1 or claim 2, wherein rapamycin is administered via gastrointestinal administration or via parenteral administration.

14. The method of claim 1 or claim 2, wherein rapamycin is administered via oral, gavage or rectal administration.

15. The method of claim 1 or claim 2, wherein rapamycin is administered via intravenous, intramuscular, intranasal, intraperitoneal, or subcutaneous administration.

16. Rapamycin for use in the treatment of toxic shock.

17. The use of claim 16, wherein the toxic shock is induced by exposure to a Staphylococcal exotoxin.

18. The use of claim 16, wherein the toxic shock is induced by exposure to a toxin selected from the group consisting of Staphylococcal enterotoxin A (SEA), Staphylococcal enterotoxin B (SEB), toxic shock syndrome toxin 1 (TSST-1), streptococcal pyrogenic exotoxin A (SPEA), and streptococcal pyrogenic exotoxin C (SPEC).

19. Use of rapamycin for the manufacture of a medicament for the treatment of toxic shock.

20. The use of claim 19, wherein the toxic shock is induced by exposure to a Staphylococcal exotoxin.

21. The use of claim 19, wherein the toxic shock is induced by exposure to a toxin selected from the group consisting of Staphylococcal enterotoxin A (SEA), Staphylococcal enterotoxin B (SEB), toxic shock syndrome toxin 1 (TSST-1), streptococcal pyrogenic exotoxin A (SPEA), and streptococcal pyrogenic exotoxin C (SPEC).
